(19) United States
(12) Patent Application Publication
(10) Pub. No.: US 2025/0283864 A1
(43) Pub. Date: Sep. 11, 2025

(54) Combined In Vitro Prediction Method for Glycemic Index of Liquid Food for Special Medical Purpose

(71) Applicant: Wuxi Institute For Food Control, Wuxi (CN)

(72) Inventors: Mi ZHANG, Wuxi (CN); Xiaodong HUANG, Wuxi (CN); Yongwei FENG, Wuxi (CN); Peiyao LU, Wuxi (CN); Zihao FAN, Wuxi (CN); Feng ZHANG, Wuxi (CN)

(21) Appl. No.: 19/034,988

(22) Filed: Jan. 23, 2025

(30) Foreign Application Priority Data
Mar. 11, 2024 (CN) 202410272534.3

(51) Int. Cl. G01N 33/10; G01N 1/28

(52) U.S. Cl. CPC G01N 33/10; G01N 1/28

(57) ABSTRACT

The disclosure discloses a combined in vitro prediction method for a glycemic index of liquid FSMPs. The method includes the following steps: simulating an optimal digestion condition when liquid FSMPs passes through an oral cavity, a stomach, and a small intestine; detecting a generation quantity of glucose in the liquid FSMPs in real time to predict an in vitro GI value of the liquid FSMPs; detecting a degree of hydrolysis of starch in the sample in real time to predict a theoretical GI value of the liquid FSMPs; and predicting the glycemic index of the liquid FSMPs by calculating a mean value of the in vitro GI value and the theoretical GI value and analyzing a correlation of the in vitro GI value and the theoretical GI value.

The disclosure relates to the technical field of glycemic index detection for foods, and particularly relates to a combined in vitro prediction method for a glycemic index of liquid foods for special medical purposes.

### BACKGROUND

Foods for special medical purposes (FSMPs) are formula foods specially processed and prepared to meet special demands of various crowds with special diseases on nutrients or diets. Therefore, reasonably evaluating digestion and absorption of the foods for special medical purposes in a human body is crucial to the actual functional characteristics of a product. The glycemic index (GI), as an index for describing the digestion and absorption rate of foods by the human body and reflecting the blood glucose elevating degree of the human body after eating, has aroused wide concern among researchers.

At present, the GI value of the food is detected in countries or regions still taking a human body postprandial blood glucose test specification ISO 26642:2010 published by the international organization for standardization (ISO) as a universal standard. Based on this standard, a recommended standard (WS/T 652-2019 Standard for determination of food glycemic index) has been published in China in 2019, and the test method is consistent with that in ISO 26642:2010. Although an in vivo blood glucose test determines the GI value of the food in a real digestive environment of a human body, the in vivo blood glucose test is not suitable for enterprises to screen high-throughput and low-GI food raw materials and research, develop, and promote low-GI foods because the in vivo blood glucose test needs at least 12 volunteers for parallel experiments, which not only involves corresponding ethical applications, but also is expensive and low in efficiency, and experimental results are significantly affected by individual differences, so that the application range of the in vivo blood glucose test is greatly limited.

In order to overcome a series of problems existing in the in vivo blood glucose test, it is an urgent need to develop a rapid and simple in vitro prediction method for fast evaluation of the GI value of a liquid food sample for special medical purposes.

### SUMMARY

To solve the problems in the prior art, the disclosure provides a combined in vitro prediction method for a glycemic index of liquid foods for special medical purposes, to solve the technical problems that current GI value in vivo determination is time-consuming, complex in experimental process, low in efficiency, and the like.

Therefore, the specific technical solution used by the disclosure is as follows:

A combined in vitro prediction method for a glycemic index of liquid foods for special medical purposes, including the following steps:

S1, analyzing and obtaining an optimal digestion condition parameter based on a digestion parameter optimization algorithm, and simulating an optimal digestion condition when a liquid food sample for special medical purposes passes through an oral cavity, a stomach, and a small intestine;
S2, detecting a generation quantity of glucose in the liquid food sample for special medical purposes in real time by a glucose analyzer to predict an in vitro GI value of the liquid food sample for special medical purposes;
S3, detecting a degree of hydrolysis of starch in the sample in real time through a 3,5-dinitrosalicylic acid method to predict a theoretical GI value of the liquid food sample for special medical purposes; and
S4, predicting the glycemic index of the liquid foods for special medical purposes by calculating a mean value of the in vitro GI value and the theoretical GI value and analyzing a correlation of the in vitro GI value and the theoretical GI value.

Further, the analyzing and obtaining an optimal digestion condition parameter based on a digestion parameter optimization algorithm, and simulating an optimal digestion condition when a liquid food sample for special medical purposes passes through an oral cavity, a stomach, and a small intestine include the following steps:
S11, determining optimization objectives in three different digestion stages: oral cavity, stomach, and small intestine, and analyzing and identifying control variables that affect the digestion stages;
S12, dividing time of each digestion stage into multiple time periods corresponding to different control variables, and generating an input vector group for each control variable according to a time discretization result;
S13, setting a size of a group and a number of iterations, determining a crossover rate and a mutation strategy, and performing optimization calculation on a digestion condition parameter combination by the digestion parameter optimization algorithm to seek for an optimal digestion condition parameter combination for each digestion stage; and
S14, simulating digestion conditions when the liquid food sample for special medical purposes passes through the oral cavity, the stomach, and the small intestine based on the optimal digestion condition in the optimal digestion condition parameter combination.

Further, the dividing time of each digestion stage into multiple time periods corresponding to different control variables, and generating an input vector group for each control variable according to a time discretization result include the following steps:
S121, dividing a process of each digestion stage in terms of time, and discretizing changes of the control variables to different time points according to time division of each digestion stage; and
S122, creating an input vector for a value of each control variable at each time point, and combining the input vectors of the control variables to obtain the input vector group.

Further, the setting a size of a group and a number of iterations, determining a crossover rate and a mutation strategy, and performing optimization calculation on a digestion condition parameter combination by the digestion parameter optimization algorithm to seek for an optimal digestion condition parameter combination for each digestion stage include the following steps:
S131, setting a size of a group and a number of iterations, determining a crossover rate and a mutation strategy, and designing a fitness function based on a key index;
S132, converting the digestion condition parameter in a binary encoding mode, and randomly generating a parameter combination set of an initial digestion condition;
S133, calculating a fitness value of each digestion condition parameter combination, and selecting the optimal digestion condition parameter combination according to the fitness value;
S134, performing a crossover operation on the optimal digestion condition parameter combination to generate a new digestion condition parameter combination, and performing a mutation operation on the new digestion condition parameter combination; and
S135, repeatedly executing the selecting, crossover, and mutation operations, and terminating an optimization process till a preset number of iterations is reached or the fitness meets a preset condition to obtain the optimal digestion condition parameter combination for each digestion stage.

Further, the performing a crossover operation on the optimal digestion condition parameter combination to generate a new digestion condition parameter combination, and performing a mutation operation on the new digestion condition parameter combination further include:
selecting a certain quantity of optimal digestion condition parameter combinations from each generation of the digestion condition parameter combination set, and directly reserving the optimal digestion condition parameter combinations in a new digestion condition parameter combination set; and
dividing a total digestion condition parameter combination set into multiple digestion condition parameter combination subsets, setting a rule, and allowing migration of the digestion condition parameter combinations among the plurality of digestion condition parameter combination subsets.

Further, the detecting a generation quantity of glucose in the liquid food sample for special medical purposes in real time by a glucose analyzer to predict an in vitro GI value of the liquid food sample for special medical purposes includes the following steps:
S21, placing the liquid food sample for special medical purposes in a reactor, adding a stirring rotor in the reactor, and setting corresponding stirring temperature and speed according to the parameters in the optimal digestion condition;
S22, simulating the digestion condition by using an artificial gastrointestinal simulator, where the artificial gastrointestinal simulator automatically adds α-amylase, pepsin, a sodium hydroxide solution, an acetic acid solution, and a trypsin mixed solution at intervals into the reactor filled with the liquid food sample for special medical purposes in sequence to react to obtain digestive juice, the trypsin mixed solution including trypsin and amyloglucosidase; and
S23, detecting a glucose content in the digestive juice by the glucose analyzer, and calculating the in vitro GI value of the liquid food sample for special medical purposes by built-in software of the glucose analyzer.

Further, the trypsin mixed solution includes trypsin and amyloglucosidase;
a concentration of the α-amylase is 100-1000 U/ml; and/or,
a concentration of the pepsin is 1-10 mg/ml; and/or,
a concentration of the trypsin is 1-10 mg/ml; and/or,
a concentration of the amyloglucosidase is 1-10 mg/ml; and/or,
a concentration of the sodium hydroxide solution is 1-5 mol/L; and/or,
a concentration of the acetic acid solution is 1-5 mol/L;
an additive amount of the α-amylase is 1-5 mL; and/or,
an additive amount of the pepsin is 1-10 mL; and/or,
an additive amount of the trypsin mixed solution is 1-10 ml; and/or,
an additive amount of the sodium hydroxide solution is 1-10 ml; and/or,
an additive amount of the acetic acid solution is 10-30 mL.

Further, the detecting a degree of hydrolysis of starch in the sample in real time through a 3,5-dinitrosalicylic acid method to predict a theoretical GI value of the liquid food sample for special medical purposes includes the following steps:
S31, drawing a standard curve: taking D-glucose anhydrous as a standard substance,
adding water to dissolve the standard substance and dilute to a certain volume to obtain a mass concentration of the standard substance, then adding water with different volumes to dilute the standard substance to obtain solutions with different mass concentrations, taking the solutions with different mass concentrations, adding a 3,5-dinitrosalicylic acid reagent respectively, shaking the solutions well, performing color development by a water bath, cooling the solutions, adding water to dilute to a certain volume, and detecting an absorbance, taking distilled water as a blank control, to obtain the standard curve taking the mass concentration as a horizontal coordinate and the absorbance as a vertical coordinate;
S32, simulating the digestion: placing the liquid food sample for special medical purposes in the reactor, adding a stirring rotor in the reactor, setting stirring temperature and speed according to the parameters in the optimal digestion condition, adding α-amylase and oral digestive juice, performing constant temperature water bath oscillation, then adding the pepsin, adjusting a pH value of the digestive juice with a hydrochloric acid solution, adding gastric digestive juice, performing constant temperature water bath oscillation, then adding trypsin, adjusting a pH value with the sodium hydroxide solution, adding intestinal digestive juice, performing constant temperature water bath oscillation to obtain the digestive juice, taking the digestive juice at different time points, adding the 3,5-dinitrosalicylic acid reagent, detecting an absorbance, and obtaining a mass concentration of glucose in the digestive juice through the absorbance and the standard curve; and
S33, determining a total starch content in the sample and calculating a theoretical GI value result: placing the liquid food sample for special medical purposes in a centrifuge tube, adding the hydrochloric acid solution, performing water bath and cooling, adjusting a pH value, filtering the solution, taking a filtrate, adding the 3,5-dinitrosalicylic acid reagent, detecting an absorbance, obtaining a mass concentration of glucose in the sample after starch is hydrolyzed through the absorbance and the standard curve, and then calculating and obtaining the theoretical GI value according to a formula.

Further, the oral digestive juice includes components with the following concentrations: 15-16 mM KCl, 3-4 mM KH₂PO₄, 13-14 mM NaHCO₃, 0.1-0.2 mM MgCl₂ (H₂O)₆, 0.01-0.10 mM (NH₄)₂CO₃, and 1-2 mM CaCl₂(H₂O)₂; and/or,
the gastric digestive juice includes components with the following concentrations: 5-10 mM KCl, 0.5-1.5 mM KH₂PO₄, 20-30 mM NaHCO₃, 40-50 mM NaCl, 0.1-0.3 mM MgCl₂ (H₂O)₆, 0.1-1.0 mM (NH₄)₂CO₃, and 0.1-0.2 mM CaCl₂) (H₂O)₂; and/or,
the intestinal digestive juice includes components with the following concentrations: 5-10 mM KCl, 0.1-1 mM KH₂PO₄, 50-100 mM NaHCO₃, 30-50 mM NaCl, 0.1-1.0 mM MgCl₂ (H₂O)₆, and 0.1-1.0 mM CaCl₂) (H₂O)₂;
a concentration of the α-amylase is 50-200 U/ml; and/or,
a concentration of the pepsin is 1000-3000 U/ml; and/or,
a concentration of the trypsin is 50-200 U/ml; and/or,
a concentration of the sodium hydroxide solution is 0.1-2.0 M; and/or,
a concentration of the hydrochloric acid solution is 0.1-2.0 M;
an additive amount of the α-amylase is 1-5 mL; and/or,
an additive amount of the pepsin is 1-10 ml; and/or,
an additive amount of the trypsin is 10-20 mL.

Further, in step S33, a calculation formula for the hydrolysis rate of starch (HRS) is as follows:

$$HRS = [ ( {m}_{1} \times 0.9 ) / m ] \times 100$$

where m is the total starch content, in mg; m₁ is an amount of glucose in the digestive juice, in mg; and 0.9 is a conversion coefficient for glucose and starch;
a hydrolysis curve is drawn taking time as a horizontal coordinate and the HRS as a vertical coordinate, a hydrolysis index (HI) of starch in the sample during digestion is calculated, and a calculation formula for the HI of starch in the sample during digestion is as follows:

$$HI = ( {AUC}_{sample} / {AUC}_{glucose standard substance} ) \times 100$$

where AUCₛₐₘₚₗₑ is an area under a starch hydrolysis curve of the sample; and AUC_{glucose} standard substance is an area under a standard starch hydrolysis curve; and
a calculation formula for the glycemic index is as follows:

$$GI = 39.71 + 0.549 \times HI .$$

The disclosure has the following beneficial effects:

1) The disclosure provides a combined in vitro prediction method for a glycemic index of liquid foods for special medical purposes, which can predict the GI value of the food more rapidly and efficiently by in vitro simulating oral chewing and digestion and absorption processes of stomach and small intestine of the human body, and may be used for screening raw materials of liquid foods for special medical purposes or formula foods on a large scale in the initial period.

2) The combined in vitro prediction method provided by the disclosure includes an automatic digestion simulation method based on a GI20 instrument (for predicting the in vitro GI value) and a lab digestion simulation method based on a hydrolysis rate of starch (for predicting the theoretical GI value). The two simulation modes can effectively improve the effectiveness and stability of a predicted result for combined prediction of the GI values (including the in vitro value and the theoretical value).

3) According to the disclosure, the optimal digestion condition parameter combination suitable for different digestion stages may be effectively found by the digestion parameter optimization algorithm, so that the digestion condition when the liquid food sample for special medical purposes passes through the oral cavity, the stomach, and the small intestine may be simulated based on the optimal digestion condition in the optimal digestion condition parameter combination. Therefore, it may be closer to a real digestion process of the human body, so that the digestion efficiency and effect of the foods for special medical purposes in the whole digestion process are optimized.

### BRIEF DESCRIPTION OF FIGURES

FIG. 1 is a flowchart of a combined in vitro prediction method for a glycemic index of liquid foods for special medical purposes according to an example of the disclosure;

FIG. 2 is a glucose generation curve graph in an example 1 of the disclosure; and

FIG. 3 is a curve graph of a hydrolysis rate of starch in the example 1 of the disclosure.

### DETAILED DESCRIPTION

In order to make purposes, technical solutions and advantages of the disclosure more clearly, the technical solutions in the examples of the disclosure will be clearly and completely described below. Examples with unmarked specific conditions below generally follow conventional conditions or conditions suggested by manufacturers. The used reagents or instruments not indicated by manufacturers are conventional products which are available on the market. Besides, the meaning of “and/or” in the disclosure includes three parallel schemes. By taking “A and/or B” as an example, it includes a scheme A or a scheme B or a scheme meeting A and B simultaneously. In addition, the technical solutions of various examples may be combined one another based on implementation by persons of ordinary skill in the art. When the technical solutions contradict each other in combination or may not be implemented, it is to be considered that there is no such combination of the technical solutions, which shall not fall into the protection scope of the disclosure. On the basis of the examples in the disclosure, all other examples obtained by persons of ordinary skill in the art without making creative efforts fall into the scope of protection of the disclosure.

According to the example of the disclosure, provided is a combined in vitro prediction method for a glycemic index of liquid foods for special medical purposes.

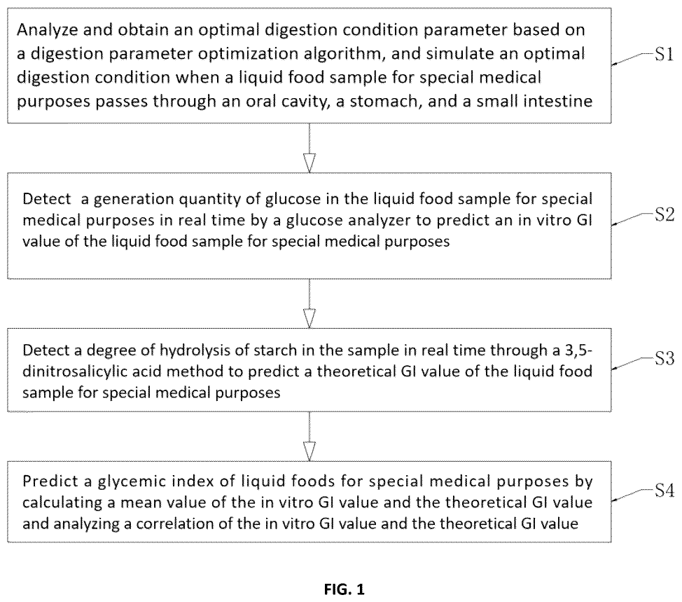

The disclosure is further described now in conjunction with drawings and specific implementations. As shown in FIG. 1, a combined in vitro prediction method for a glycemic index of liquid foods for special medical purposes according to the example of the disclosure includes the following steps:

S1, an optimal digestion condition parameter is analyzed and obtained based on a digestion parameter optimization algorithm, and an optimal digestion condition when a liquid food sample for special medical purposes passes through an oral cavity, a stomach, and a small intestine is simulated.

Simulating the digestion conditions when the liquid food sample for special medical purposes passes through the oral cavity, the stomach, and the small intestine is a complex process, and when the digestion conditions are simulated, it is necessary to consider specific characteristics of the liquid foods for special medical purposes, such as components, concentration, and texture because these factors will affect the digestion process. By simulating these digestion conditions, the digestion process of the food in the human body may be evaluated more accurately, so that the influence of the digestion process on blood glucose level is predicted.

Simulating the digestion process involves in many parameters such as temperature, pH value, and type and concentration of enzymes. There may be complex interactions among these parameters, so that it is quite important to find the optimal parameter combination. Therefore, the optimal digestion condition parameter is analyzed and obtained based on the digestion parameter optimization algorithm in the disclosure, so that the optimal digestion condition when a liquid food sample for special medical purposes passes through an oral cavity, a stomach, and a small intestine may be simulated.

The analyzing and obtaining an optimal digestion condition parameter based on a digestion parameter optimization algorithm, and simulating an optimal digestion condition when a liquid food sample for special medical purposes passes through an oral cavity, a stomach, and a small intestine include the following steps:
S11, determining optimization objectives in three different digestion stages: oral cavity, stomach, and small intestine, for example, maximizing nutrient absorption, minimizing discomfort or optimizing digestive efficiency, and analyzing and identifying control variables that affect the digestion stages, for example, temperature, pH value, enzyme activity, and food composition;
S12, dividing time of each digestion stage into multiple time periods corresponding to different control variables, and generating an input vector group for each control variable according to a time discretization result, where specifically, the dividing time of each digestion stage into multiple time periods corresponding to different control variables, and generating an input vector group for each control variable according to a time discretization result include the following steps:
S121, dividing a process of each digestion stage in terms of time, for example, dividing the oral cavity stage into time periods of food chewing and primary digestion, dividing the stomach stage into time periods of food mixing and preliminary decomposition, and dividing the small intestine stage into time periods of nutrient absorption and residue treatment; and determining the control variables in the digestion process, setting a value range and a level for each control variable, and discretizing the control variables to different time points according to time division of each digestion stage; and
S122, creating an input vector for a value of each control variable at each time point, where this vector represents the change of the control variable in the whole digestion process, and combining the input vectors of the control variables to obtain a large input vector group, where this vector group will serve as input data of a genetic algorithm;
S13, setting a size of a group and a number of iterations, determining a crossover rate and a mutation strategy, and performing optimization calculation on a digestion condition parameter combination by the digestion parameter optimization algorithm to seek for an optimal digestion condition parameter combination for each digestion stage, where
specifically, the setting a size of a group and a number of iterations, determining a crossover rate and a mutation strategy, and performing optimization calculation on a digestion condition parameter combination by the digestion parameter optimization algorithm to seek for an optimal digestion condition parameter combination for each digestion stage include the following steps:
S131, determining a size of a group in the algorithm, where this size shall be large enough to ensure enough diversity, but shall also consider limitation of a calculation resource; setting the number of iterations of algorithm operation, where this numerical value needs to be determined according to an expected optimization level and acceptable calculation time; determining a proportion of new individuals generated by cross in the genetic algorithm, where a proper crossover rate contributes to effectively exploring solution space; determining a mode and a frequency of mutation, where the mutation contributes to introducing new features and preventing premature convergence of the algorithm; and selecting key indexes capable of reflecting the digestive efficiency and quality, for example, nutrient substance release rate and digestion time, and based on these indexes, constructing a fitness function to evaluate effects of different parameter combinations;
S132, converting the digestion condition parameter in a binary encoding mode, and randomly generating a parameter combination set of an initial digestion condition, where an individual in the parameter combination set for each digestion condition represents a parameter combination that may obtain the digestion conditions;
S133, calculating a fitness value of each digestion condition parameter combination, and selecting the optimal digestion condition parameter combination according to the fitness value to ensure that excellent features can be reserved and transferred;
S134, performing a crossover operation on the optimal digestion condition parameter combination to generate a new digestion condition parameter combination, so as to explore a new parameter combination, and performing a mutation operation on the new digestion condition parameter combination to introduce a new heritable mutation, so as to increase the diversity of the group; and
S135, repeatedly executing the selecting, crossover, and mutation operations, and terminating an optimization process till a preset number of iterations is reached or the fitness meets a preset condition to obtain the optimal digestion condition parameter combination for each digestion stage, where
in addition, the performing a crossover operation on the optimal digestion condition parameter combination to generate a new digestion condition parameter combination, and performing a mutation operation on the new digestion condition parameter combination further include:
selecting a certain quantity of optimal digestion condition parameter combinations from each generation of the digestion condition parameter combination set, and directly reserving the optimal digestion condition parameter combinations in a new digestion condition parameter combination set; and
dividing a total digestion condition parameter combination set into multiple digestion condition parameter combination subsets, setting a rule, and allowing migration of the digestion condition parameter combinations among the plurality of digestion condition parameter combination subsets; and
S14, simulating digestion conditions when the liquid food sample for special medical purposes passes through the oral cavity, the stomach, and the small intestine based on the optimal digestion condition in the optimal digestion condition parameter combination.

S2, a generation quantity of glucose in the liquid food sample for special medical purposes is detected in real time by a glucose analyzer to predict an in vitro GI value of the liquid food sample for special medical purposes,
where the detecting a generation quantity of glucose in the liquid food sample for special medical purposes in real time by a glucose analyzer to predict an in vitro GI value of the liquid food sample for special medical purposes includes the following steps:
S21, placing the liquid food sample for special medical purposes in a reactor, adding a stirring rotor in the reactor, and setting corresponding stirring temperature and speed according to the parameters in the optimal digestion condition;
S22, simulating the digestion condition by using an artificial gastrointestinal simulator, where the artificial gastrointestinal simulator automatically adds α-amylase, pepsin, a sodium hydroxide solution, an acetic acid solution, and a trypsin mixed solution (the trypsin mixed solution includes trypsin and amyloglucosidase) at intervals into the reactor filled with the liquid food sample for special medical purposes in sequence to react to obtain digestive juice, where
specifically, the trypsin mixed solution includes trypsin and amyloglucosidase;
a concentration of the α-amylase is 100-1000 U/ml; and/or,
a concentration of the pepsin is 1-10 mg/ml; and/or,
a concentration of the trypsin is 1-10 mg/ml; and/or,
a concentration of the amyloglucosidase is 1-10 mg/ml; and/or,
a concentration of the sodium hydroxide solution is 1-5 mol/L; and/or,
a concentration of the acetic acid solution is 1-5 mol/L;
an additive amount of the α-amylase is 1-5 mL; and/or,
an additive amount of the pepsin is 1-10 mL; and/or,
an additive amount of the trypsin mixed solution is 1-10 mL; and/or,
an additive amount of the sodium hydroxide solution is 1-10 mL; and/or,
an additive amount of the acetic acid solution is 10-30 mL; and
S23, detecting a glucose content in the digestive juice by the glucose analyzer, and calculating the in vitro GI value of the liquid food sample for special medical purposes by built-in software of the glucose analyzer.
S3, a degree of hydrolysis of starch in the sample is detected in real time through a 3,5-dinitrosalicylic acid method to predict a theoretical GI value of the liquid food sample for special medical purposes,
where the detecting a degree of hydrolysis of starch in the sample in real time through a 3,5-dinitrosalicylic acid method to predict a theoretical GI value of the liquid food sample for special medical purposes includes the following steps:
S31, drawing a standard curve: taking D-glucose anhydrous as a standard substance, adding water to dissolve the standard substance and dilute to a certain volume to obtain a mass concentration of the standard substance, then adding water with different volumes to dilute the standard substance to obtain solutions with different mass concentrations, taking the solutions with different mass concentrations, adding a 3,5-dinitrosalicylic acid reagent respectively, shaking the solutions well, performing color development by a water bath, cooling the solutions, adding water to dilute to a certain volume, and detecting an absorbance, taking distilled water as a blank control, to obtain the standard curve taking the mass concentration as a horizontal coordinate and the absorbance as a vertical coordinate;
S32, simulating the digestion: placing the liquid food sample for special medical purposes in the reactor, adding a stirring rotor in the reactor, setting stirring temperature and speed according to the parameters in the optimal digestion condition, adding α-amylase and oral digestive juice, performing constant temperature water bath oscillation, then adding the pepsin, adjusting a pH value of the digestive juice with a hydrochloric acid solution, adding gastric digestive juice, performing constant temperature water bath oscillation, then adding trypsin, adjusting a pH value with the sodium hydroxide solution, adding intestinal digestive juice, performing constant temperature water bath oscillation to obtain the digestive juice, taking the digestive juice at different time points, adding the 3,5-dinitrosalicylic acid reagent, detecting an absorbance, and obtaining a mass concentration of glucose in the digestive juice through the absorbance and the standard curve, where
specifically, the oral digestive juice includes components with the following concentrations: 15-16 mM KCl, 3-4 mM KH₂PO₄, 13-14 mM NaHCO₃, 0.1-0.2 mM MgCl₂ (H₂O)₆, 0.01-0.10 mM (NH₄)₂CO₃, and 1-2 mM CaCl₂) (H₂O)₂; and/or,
the gastric digestive juice includes components with the following concentrations: 5-10 mM KCl, 0.5-1.5 mM KH₂PO₄, 20-30 mM NaHCO₃, 40-50 mM NaCl, 0.1-0.3 mM MgCl₂ (H₂O)₆, 0.1-1.0 mM (NH₄)₂CO₃, and 0.1-0.2 mM CaCl₂) (H₂O)₂; and/or,
the intestinal digestive juice includes components with the following concentrations: 5-10 mM KCl, 0.1-1 mM KH₂PO₄, 50-100 mM NaHCO₃, 30-50 mM NaCl, 0.1-1.0 mM MgCl₂ (H₂O)₆, and 0.1-1.0 mM CaCl₂) (H₂O)₂;
a concentration of the α-amylase is 50-200 U/mL; and/or,
a concentration of the pepsin is 1000-3000 U/ml; and/or,
a concentration of the trypsin is 50-200 U/mL; and/or,
a concentration of the sodium hydroxide solution is 0.1-2.0 M; and/or,
a concentration of the hydrochloric acid solution is 0.1-2.0 M;
an additive amount of the α-amylase is 1-5 mL; and/or,
an additive amount of the pepsin is 1-10 ml; and/or,
an additive amount of the trypsin is 10-20 mL; and
S33, determining a total starch content in the sample and calculating a theoretical GI value result: placing the liquid food sample for special medical purposes in a centrifuge tube, adding the hydrochloric acid solution, performing water bath and cooling, adjusting a pH value, filtering the solution, taking a filtrate, adding the 3,5-dinitrosalicylic acid reagent, detecting an absorbance, obtaining a mass concentration of glucose in the sample after starch is hydrolyzed through the absorbance and the standard curve, and then calculating and obtaining the theoretical GI value according to a formula.

Specifically, in step S33, a calculation formula for the hydrolysis rate of starch (HRS) is as follows:

$$HRS = [ ( {m}_{1} \times 0.9 ) / m ] \times 100$$

where m is the total starch content, in mg; m₁ is an amount of glucose in the digestive juice, in mg; and 0.9 is a conversion coefficient for glucose and starch;
a hydrolysis curve is drawn taking time as a horizontal coordinate and the HRS as a vertical coordinate, a hydrolysis index (HI) of starch in the sample during digestion is calculated, and a calculation formula for the HI of starch in the sample during digestion is as follows:

$$HI = ( {AUC}_{sample} / {AUC}_{glucose standard substance} ) \times 100$$

where AUCₛₐₘₚₗₑ is an area under a starch hydrolysis curve of the sample; and AUC_{glucose standard substance} is an area under a standard starch hydrolysis curve; and
a calculation formula for the glycemic index (GI) is as follows:

$$GI = 39.71 + 0.549 \times HI .$$

S4, the glycemic index of the liquid foods for special medical purposes is predicted by calculating a mean value of the in vitro GI value and the theoretical GI value and analyzing a correlation of the in vitro GI value and the theoretical GI value.

In addition, in order to facilitate understanding of the above technical solutions of the disclosure, specific examples of the disclosure in the actual process are described in detail below.

### Example 1

### Optimal Digestion Condition Parameter Analysis

In this example, digestion of a special liquid foods for special medical purposes was simulated. First, the main components (20% by mass of proteins, 55% of carbohydrates and 25% of fat) of the food were analyzed. Then, optimization objectives were set according to this information, nutrient absorption was maximized, and the digestion comfort was considered as well. Next, the digestion process was divided into three stages: oral cavity, stomach, and small intestine, and each stage was analyzed in detail to determine key control variables that affected digestion in these stages. For the oral cavity stage, the temperature was normal temperature (about 37° C.), the pH value as 6.8, the activity of sialidase was moderate (a simulation value was set as an activity index 5, assuming that the range was 1-10), the enzyme activity in saliva was set as 5 (the definition range was 1-10), the standing time of the food in the oral cavity was set as 30 s to 2 min, and this time range allowed full chewing and saliva mixing to simulate the chewing demands on different types of foods; for the stomach, the temperature was about 37° C., the pH value was 2.0 (a strong acid environment), and the stomach peristaltic intensity is high (a simulation value was set as an intensity index 8, assuming that the range was 1-10); and for the small intestine, the temperature was about 37° C., the pH value was 7.5 (a slightly alkaline environment), the enzyme activity in the intestinal tract was high (a simulation value was set as an activity index 9, assuming that the range was 1-10), a passing rate of the food was set as 2-6 h, and this time range reflected differences of different types of foods and digestive efficiencies. Then, the digestion parameter optimization algorithm was used to seek for the optimal condition parameter combination for each digestion stage. The proper size of the group was set as 100 individuals, the number of iterations was 50 generations, the crossover rate was 0.8 (80% of the individuals generated new individuals by cross), and the mutation rate was 0.1 (10% of the new individuals experienced mutation), and a fitness function was constructed to reflect the nutrient absorption efficiency and the digestive efficiency.

An expression of the fitness function is as follows:

$$F ( x ) = {w}_{1} \times N ( x ) + {w}_{2} \times D ( x ) + {w}_{3} \times G ( x ) + {w}_{4} \times E ( x ) + {w}_{5} \times T ( x )$$

where F(x) represents a fitness function value in a given digestion condition parameter combination x;
N(x) represents a nutrient absorption rate in the given digestion condition parameter combination x;
D(x) represents the efficiency in the whole digestion process, including the decomposition rate and the completeness of the food;
G(x) represents evaluation of a digestive discomfort level in the given digestion condition parameter combination x;
E(x) represents an effect of an optimized adjustment on the enzyme activities in saliva and intestinal tract;
T(x) represents quantification of an optimization effect of the food at standing time in each digestion stage, including the oral cavity stage, the stomach stage, and the small intestine stage; and
w₁, w₂, w₃, w₄, and w₅ are weight coefficients of the indexes for adjusting relative importance of the indexes in total fitness evaluation.

The optimal digestion condition parameter combination was sought for through the iterative optimization process:

### Oral Cavity:

The temperature was 37° C. to simulate the conditions under the normal body temperature of the human body.

The pH value was 6.8, and this value was the pH value of the oral cavity in a natural state, which was beneficial for the activity of the sialidase.

Activity index of the sialidase was adjusted to 6 (it was assumed as 5 previously), and it was found by the optimization algorithm that slightly improving the enzyme activity could more effectively start the decomposition process of the carbohydrates.

Standing time of the food in the oral cavity was optimized to 1 min, which was the most suitable for fully chewing and saliva mixing, so as to improve the efficiency of primary digestion of the food.

### Stomach Stage:

The temperature was kept at 37° C., which met the normal operation temperature of the stomach of the human body.

The pH value was optimized to 1.8 (it was assumed as 2.0 previously), and the lower pH value was beneficial for rapid decomposition of proteins.

The peristaltic intensity index of the stomach was maintained at 8 to ensure the efficiency of food mixing and preliminary decomposition.

Small intestine stage:

The temperature was kept at 37° C., which was suitable for the nutrient absorption process of the small intestine.

The pH value was adjusted to 7.6 (it was assumed as 7.5 previously), and the slightly alkaline environment was more beneficial for the activities and functions of the enzymes in the intestinal tract.

The activity index of the enzymes in the intestinal tract was optimized to 10 (maximum value) to ensure that nutritional substances were decomposed and absorbed to the maximum extent.

The passing speed of the food was optimized to 4 h, this time was the most suitable for maximizing nutrient absorption, and the digestion comfort was considered as well.

Finally, the digestion process of the liquid foods for special medical purposes in the oral cavity, the stomach, and the small intestine was simulated by using these optimized parameters.

### Example 2

### (1) Prediction of an In Vitro GI Value

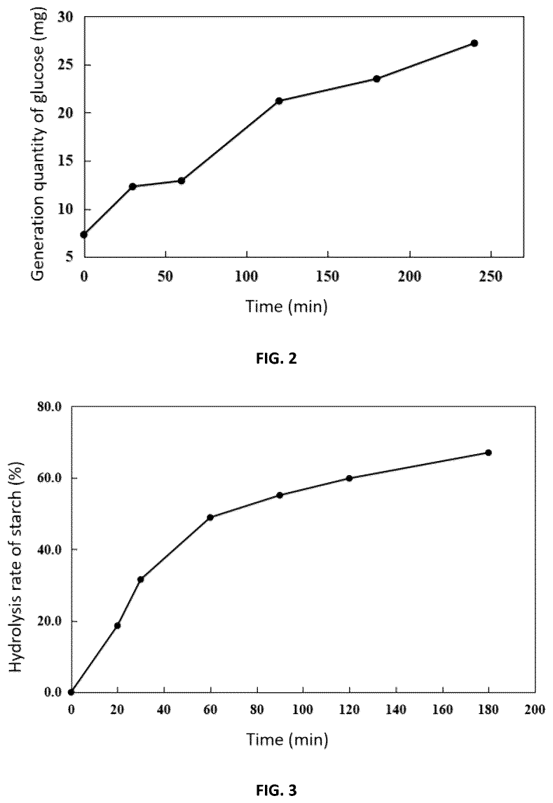

A sample with a proper mass was weighed and placed in a sample cup, where the sample contained approximately 40 mg of digestible carbohydrates. A magnetic stirring rotor was added in each sample cup and the sample cup was placed at a corresponding position. A sample stirring temperature was set as 30° C., a stirring rate was set as 100 rpm/min, a start button was clicked in built-in software of a glucose analyzer to start measurement to run a measuring program. After the measuring program started, an artificial gastrointestinal simulator added 1 mL of α-amylase (100 U/mL) into each sample cup automatically; after operation of the instrument for 1 min, 1 mL of pepsin (1 mg/ml) was continuously added; after operation of the instrument for 10 min, 1 mL of a sodium hydroxide solution with a concentration of 1 M was added; after operation of the instrument for 5 min, 10 mL of an acetic acid solution with a concentration of 1 M was added into each sample; in 5 min, 1 mL of a trypsin mixed solution (including 1 mg/ml trypsin and 1 mg/mL amyloglucosidase) was added, and then at every 1 h, the instrument automatically took 2 mL of digestive juice from the sample cup to detect the glucose content (totally measured for 2 h) to obtain FIG. 2. The built-in software of the glucose analyzer calculated the in vitro GI value of the sample.

### (2) Prediction of a Theoretical GI Value

Standard curve: 300 mg of D-glucose anhydrous was weighed as a standard substance, and water was added to dissolve the standard substance and dilute to 200 mL. The mass concentration of the solution was 1.5 mg/mL. Then water was added to dilute the solution to 0.075 mg/ml, 0.150 mg/mL, 0.300 mg/mL, 0.600 mg/mL, 0.750 mg/mL, 1.000 mg/mL, and 1.500 mg/ml to obtain a series of mass concentration solutions. 1 ml of the solution was taken, 1 mL of a 3, 5-dinitrosalicylic acid reagent was added, the mixture was shaken well, water bath was performed at 80° C. to develop for 5 min, and after being taken out, the sample was cooled to room temperature by flowing water, water was added to dilute to a certain volume, the mixture was shaken well, the absorbance was detected at 500 nm wavelength taking distilled water as a blank control, and the standard curve was drawn taking the mass concentration (C, mg/ml) as a horizontal coordinate and the absorbance (A) as a vertical coordinate.

Digestion simulation: the sample stirring temperature was set as 30° C., and the stirring speed was 100 rpm/min; 2 mL of the sample was sucked, 1 mL of α-amylase (50 U/mL) was added, and the mixture was subjected to constant temperature water bath oscillation for 1 min; after oral digestion was simulated, 1 mL of pepsin (1000 U/mL) was added into the oral digestive juice, the pH value of the digestive juice was adjusted to 3.0 with a hydrochloric acid solution (0.1 M), and the digestive juice was subjected to constant temperature water bath oscillation for digestion for 1 h; after gastric digestion was simulated, 10 mL of trypsin (50 U/ml) was added into the gastric digestive juice, and the pH value of the digestive juice was adjusted to 7.0 with a sodium hydroxide solution (0.1 M). In the digestion process, at every 30 min, 1 mL of the digestive juice was sucked, and the glucose contents in the digestive juice at different time points were determined by the 3,5-dinitrosalicylic acid method (totally measured for 2 h).

The oral digestive juice that simulates oral digestion includes the following components: KCl (15 mM), KH₂PO₄ (3 mM), NaHCO₃ (13 mM), MgCl₂ (H₂O)₆ (0.1 mM), (NH₄)₂CO₃ (0.01 mM), and CaCl₂) (H₂O)₂ (1 mM); the gastric digestive juice that simulates gastric digestion includes the following components: KCl (5 mM), KH₂PO₄ (0.5 mM), NaHCO₃ (20 mM), NaCl (40 mM), MgCl₂ (H₂O)₆ (0.1 mM), (NH₄)₂CO₃ (0.1 mM), and CaCl₂) (H₂O)₂ (0.1 mM); and the intestinal digestive juice that simulates intestinal digestion includes the following components: KCl (5 mM), KH₂PO₄ (0.1 mM), NaHCO₃ (50 mM), NaCl (30 mM), MgCl₂ (H₂O)₆ (0.1 mM), and CaCl₂) (H₂O)₂ (0.1 mM).

Determination of the total starch content in the sample and determination of the theoretical GI value of the sample: a proper amount of sample was placed in a centrifuge tube, 5 mL of a hydrochloric acid solution with a concentration of 5 M was added, the sample was subjected to water bath at 80° C. for 5 min, the sample was cooled to room temperature by flowing water, the pH value was adjusted to be neutral, the solution was filtered, and the glucose content in the acid hydrolyzed sample of the filtrate was determined by the 3,5-dinitrosalicylic acid method. The total starch content was calculated and obtained according to a conversion coefficient between starch and glucose (i.e., total starch content=glucose content×0.9). After the sample was digested, a curve graph of the hydrolysis rate of starch was drawn taking time as a horizontal coordinate and the hydrolysis rate of starch as a vertical coordinate (as shown in FIG. 3), and then the theoretical GI value was calculated and obtained according to a formula.

### Example 3

### (1) Prediction of an In Vitro GI Value

A sample with a proper mass was weighed and placed in a sample cup, where the sample contained approximately 60 mg of digestible carbohydrates. A magnetic stirring rotor was added in each sample cup and the sample cup was placed at a corresponding position. A sample stirring temperature was set as 35° C., a stirring rate was set as 150 rpm/min, a start button was clicked in built-in software of a glucose analyzer to start measurement to run a measuring program. After the measuring program started, the instrument added 2 mL of α-amylase (500 U/mL) into each sample cup automatically; after operation of the instrument for 2 min, 5 mL of pepsin (5 mg/mL) was continuously added; after operation of the instrument for 30 min, 5 mL of a sodium hydroxide solution with a concentration of 2 M was added; after operation of the instrument for 5 min, 20 ml of an acetic acid solution with a concentration of 2 M was added into each sample; in 5 min, 5 mL of a trypsin mixed solution (including 5 mg/mL trypsin and 5 mg/ml amyloglucosidase) was added, and then at every 1 h, the instrument automatically took 2 mL of digestive juice from the sample cup to detect the glucose content (totally measured for 4 h). The built-in software of the glucose analyzer calculated the in vitro GI value of the sample.

### (2) Prediction of a Theoretical GI Value

Standard curve: 300 mg of D-glucose anhydrous was weighed as a reference substance, and water was added to dissolve the D-glucose anhydrous and dilute to 200 mL. The mass concentration of the solution was 1.5 mg/mL. A stock solution was diluted to 0.075 mg/mL, 0.150 mg/mL, 0.300 mg/mL, 0.600 mg/mL, 0.750 mg/mL, 1.000 mg/mL, and 1.500 mg/ml to obtain a series of mass concentration solutions. 2 mL of the solution was taken, 2 mL of a 3, 5-dinitrosalicylic acid reagent was added, the mixture was shaken well, water bath was performed at 90° C. to develop for 10 min, and after being taken out, the sample was cooled to room temperature by flowing water, water was added to dilute to a certain volume, the mixture was shaken well, the absorbance was detected at 525 nm wavelength taking distilled water as a blank control, and the standard curve was drawn taking the mass concentration (C, mg/ml) as a horizontal coordinate and the absorbance (A) as a vertical coordinate.

Digestion simulation: the sample stirring temperature was set as 35° C., and the stirring speed was 150 rpm/min; 2 mL of the sample was sucked, 2 mL of α-amylase (100 U/mL) was added, and the mixture was subjected to constant temperature water bath oscillation for 2 min; after oral digestion was simulated, 5 mL of pepsin (2000 U/mL) was added into the oral digestive juice, the pH value of the digestive juice was adjusted to 3.0 with a hydrochloric acid solution (1 M), and the digestive juice was subjected to constant temperature water bath oscillation for digestion for 1.5 h; after gastric digestion was simulated, 15 ml of trypsin (100 U/mL) was added into the gastric digestive juice, and the pH value of the digestive juice was adjusted to 7.0 with a sodium hydroxide solution (1 M). In the digestion process, at every 30 min, 2 mL of the digestive juice was sucked, and the glucose contents in the digestive juice at different time points were determined by the 3,5-dinitrosalicylic acid method (totally measured for 4 h).

The oral digestive juice that simulates oral digestion includes the following components: KCl (15.5 mM), KH₂PO₄ (3.5 mM), NaHCO₃ (13.5 mM), MgCl₂ (H₂O)₆ (0.15 mM), (NH₄)₂CO₃ (0.05 mM), and CaCl₂) (H₂O)₂ (1.5 mM); the gastric digestive juice that simulates gastric digestion includes the following components: KCl (7.5 mM), KH₂PO₄ (1 mM), NaHCO₃ (25 mM), NaCl (45 mM), MgCl₂ (H₂O)₆ (0.2 mM), (NH₄)₂CO₃ (0.5 mM), and CaCl₂) (H₂O)₂ (0.15 mM); and the intestinal digestive juice that simulates intestinal digestion includes the following components: KCl (7.5 mM), KH₂PO₄ (0.5 mM), NaHCO₃ (75 mM), NaCl (40 mM), MgCl₂ (H₂O)₆ (0.5 mM), and CaCl₂) (H₂O)₂ (0.5 mM).

Determination of the total starch content in the sample and determination of the theoretical GI value of the sample: a proper amount of sample was placed in a centrifuge tube, 10 mL of a hydrochloric acid solution with a concentration of 7 M was added, the sample was subjected to water bath at 90° C. for 10 min, the sample was cooled to room temperature by flowing water, the pH value was adjusted to be neutral, the solution was filtered, and the glucose content in the acid hydrolyzed sample of the filtrate was determined by the 3,5-dinitrosalicylic acid method. The total starch content was calculated and obtained according to a conversion coefficient between starch and glucose (i.e., total starch content=glucose content×0.9). After the sample was digested, a curve graph of the hydrolysis rate of starch was drawn taking time as a horizontal coordinate and the hydrolysis rate of starch as a vertical coordinate, and then the theoretical GI value was calculated and obtained according to a formula.

### Example 4

### (1) Prediction of an In Vitro GI Value

A sample with a proper mass was weighed and placed in a sample cup, where the sample contained approximately 80 mg of digestible carbohydrates. A magnetic stirring rotor was added in each sample cup and the sample cup was placed at a corresponding position. A sample stirring temperature was set as 40° C., a stirring rate was set as 200 rpm/min, a start button was clicked in built-in software of a glucose analyzer to start measurement to run a measuring program. After the measuring program started, the instrument added 5 mL of α-amylase (1000 U/mL) into each sample cup automatically; after operation of the instrument for 5 min, 10 ml of pepsin (10 mg/mL) was continuously added; after operation of the instrument for 60 min, 10 ml of a sodium hydroxide solution with a concentration of 5 M was added; after operation of the instrument for 5 min, 30 mL of an acetic acid solution with a concentration of 5 M was added into each sample; in 5 min, 10 ml of a trypsin mixed solution (including 10 mg/ml trypsin and 10 mg/ml amyloglucosidase) was added, and then at every 1 h, the instrument automatically took 2 mL of digestive juice from the sample cup to detect the glucose content (totally measured for 6 h). The built-in software of the glucose analyzer calculated the in vitro GI value of the sample.

### (2) Prediction of a Theoretical GI Value

Standard curve: 300 mg of D-glucose anhydrous was weighed as a reference substance, and water was added to dissolve the D-glucose anhydrous and dilute to 200 mL. The mass concentration of the solution was 1.5 mg/mL. A stock solution was diluted to 0.075 mg/ml, 0.150 mg/mL, 0.300 mg/mL, 0.600 mg/mL, 0.750 mg/mL, 1.000 mg/mL, and 1.500 mg/ml to obtain a series of mass concentration solutions. 3 mL of the solution was taken, 5 mL of a 3, 5-dinitrosalicylic acid reagent was added, the mixture was shaken well, water bath was performed at 100° C. to develop for 15 min, and after being taken out, the sample was cooled to room temperature by flowing water, water was added to dilute to a certain volume, the mixture was shaken well, the absorbance was detected at 550 nm wavelength taking distilled water as a blank control, and the standard curve was drawn taking the mass concentration (C, mg/mL) as a horizontal coordinate and the absorbance (A) as a vertical coordinate.

Digestion simulation: the sample stirring temperature was set as 40° C., and the stirring speed was 200 rpm/min; 2 mL of the sample was sucked, 5 mL of α-amylase (200 U/mL) was added, and the mixture was subjected to constant temperature water bath oscillation for 5 min; after oral digestion was simulated, 10 mL of pepsin (3000 U/mL) was added into the oral digestive juice, the pH value of the digestive juice was adjusted to 3.0 with a hydrochloric acid solution (2 M), and the digestive juice was subjected to constant temperature water bath oscillation for digestion for 2 h; after gastric digestion was simulated, 20 mL of trypsin (200 U/mL) was added into the gastric digestive juice, and the pH value of the digestive juice was adjusted to 7.0 with a sodium hydroxide solution (2 M). In the digestion process, at every 30 min, 3 mL of the digestive juice was sucked, and the glucose contents in the digestive juice at different time points were determined by the 3,5-dinitrosalicylic acid method (totally measured for 6 h).

The oral digestive juice that simulates oral digestion includes the following components: KCl (16 mM), KH₂PO₄ (4 mM), NaHCO₃ (14 mM), MgCl₂ (H₂O)₆ (0.2 mM), (NH₄)₂CO₃ (0.10 mM), and CaCl₂) (H₂O)₂ (2 mM); the gastric digestive juice that simulates gastric digestion includes the following components: KCl (10 mM), KH₂PO₄ (1.5 mM), NaHCO₃ (30 mM), NaCl (50 mM), MgCl₂ (H₂O)₆ (0.3 mM), (NH₄)₂CO₃ (1.0 mM), and CaCl₂) (H₂O)₂ (0.2 mM); and the intestinal digestive juice that simulates intestinal digestion includes the following components: KCl (10 mM), KH₂PO₄ (1 mM), NaHCO₃ (100 mM), NaCl (50 mM), MgCl₂ (H₂O)₆ (1.0 mM), and CaCl₂) (H₂O)₂ (1.0 mM).

Determination of the total starch content in the sample and determination of the theoretical GI value of the sample: a proper amount of sample was placed in a centrifuge tube, 15 mL of a hydrochloric acid solution with a concentration of 10 M was added, the sample was subjected to water bath at 100° C. for 15 min, the sample was cooled to room temperature by flowing water, the pH value was adjusted to be neutral, the solution was filtered, and the glucose content in the acid hydrolyzed sample of the filtrate was determined by the 3,5-dinitrosalicylic acid method. The total starch content was calculated and obtained according to a conversion coefficient between starch and glucose (i.e., total starch content=glucose content×0.9). After the sample was digested, a curve graph of the hydrolysis rate of starch was drawn taking time as a horizontal coordinate and the hydrolysis rate of starch as a vertical coordinate, and then the theoretical GI value was calculated and obtained according to a formula.

### Comparative Example 1

Taking a sample with the known GI value as a comparative example, the GI value of the sample was obtained by in vivo blood glucose test. The GI values obtained in examples 2-4 and the comparative example 1 are shown in table 1 below.

TABLE 1
Predicted results of GI values in examples
2-4 and comparative example

| | In vitro | Theoretical | GI value | |
|---|---|---|---|---|
| Sample | GI value | GI value | (predicted) | Correlation* |
| Example 2 | 47.6 | 47.1 | 47.4 | P < 0.05 |
| Example 3 | 48.4 | 48.3 | 48.4 | P < 0.05 |
| Example 4 | 48.0 | 47.4 | 47.7 | P < 0.05 |
| | GI value (in vivo) | | | |
| Comparative | 49.0 | | | |
| example | | | | |

Remark:
*represents a correlation between the in vitro GI value and the theoretical GI value in examples

It could be known from table 1 that the results of in vitro prediction of the GI values in examples 2-4 provided by the disclosure are close to the in vivo test results, but the method tested in the disclosure is more rapid and efficient.

In conclusion, by virtue of the above technical solutions of the disclosure, the disclosure provides a combined in vitro prediction method for a glycemic index of liquid foods for special medical purposes, which can predict the GI value of the food more rapidly and efficiently by in vitro simulating oral chewing and digestion and absorption processes of stomach and small intestine of the human body, and may be used for screening raw materials of liquid foods for special medical purposes or formula foods on a large scale in the initial period. In addition, the combined in vitro prediction method provided by the disclosure includes an automatic digestion simulation method based on a GI20 instrument (for predicting the in vitro GI value) and a lab digestion simulation method based on a hydrolysis rate of starch (for predicting the theoretical GI value). The two simulation modes can effectively improve the effectiveness and stability of a predicted result for combined prediction of the GI values (including the in vitro value and the theoretical value). In addition, according to the disclosure, the optimal digestion condition parameter combination suitable for different digestion stages may be effectively found by the digestion parameter optimization algorithm, so that the digestion condition when the liquid food sample for special medical purposes passes through the oral cavity, the stomach, and the small intestine may be simulated based on the optimal digestion condition in the optimal digestion condition parameter combination. Therefore, it may be closer to a real digestion process of the human body, so that the digestion efficiency and effect of the foods for special medical purposes in the whole digestion process are optimized.

What is claimed is:

1. A combined in vitro prediction method for a glycemic index of liquid foods for special medical purposes, comprising the following steps:
S1, analyzing and obtaining an optimal digestion condition parameter based on a digestion parameter optimization algorithm, and simulating an optimal digestion condition when a liquid food sample for special medical purposes passes through an oral cavity, a stomach, and a small intestine;
S2, detecting a generation quantity of glucose in the liquid food sample for special medical purposes in real time by a glucose analyzer to predict an in vitro GI value of the liquid food sample for special medical purposes;
S3, detecting a degree of hydrolysis of starch in the sample in real time through a 3,5-dinitrosalicylic acid method to predict a theoretical GI value of the liquid food sample for special medical purposes; and
S4, predicting the glycemic index (GI) of the liquid foods for special medical purposes by calculating a mean value of the in vitro GI value and the theoretical GI value and analyzing a correlation of the in vitro GI value and the theoretical GI value.

2. The combined in vitro prediction method for a glycemic index of liquid foods for special medical purposes according to claim 1, wherein the analyzing and obtaining an optimal digestion condition parameter based on a digestion parameter optimization algorithm, and simulating an optimal digestion condition when a liquid food sample for special medical purposes passes through an oral cavity, a stomach, and a small intestine comprise the following steps:
S11, determining optimization objectives in three different digestion stages: oral cavity, stomach, and small intestine, and analyzing and identifying control variables that affect digestion stages;
S12, dividing time of each digestion stage into a plurality of time periods corresponding to different control variables, and generating an input vector group for each control variable according to a time discretization result;
S13, setting a size of a group and a number of iterations, determining a crossover rate and a mutation strategy, and performing optimization calculation on a digestion condition parameter combination by the digestion parameter optimization algorithm to seek for an optimal digestion condition parameter combination for each digestion stage; and
S14, simulating digestion conditions when the liquid food sample for special medical purposes passes through the oral cavity, the stomach, and the small intestine based on the optimal digestion condition in the optimal digestion condition parameter combination.

3. The combined in vitro prediction method for a glycemic index of liquid foods for special medical purposes according to claim 2, wherein the dividing time of each digestion stage into a plurality of time periods corresponding to different control variables, and generating an input vector group for each control variable according to a time discretization result comprise the following steps:
S121, dividing a process of each digestion stage in terms of time, and discretizing changes of the control variables to different time points according to time division of each digestion stage; and
S122, creating an input vector for a value of each control variable at each time point, and combining the input vectors of the control variables to obtain the input vector group.

4. The combined in vitro prediction method for a glycemic index of liquid foods for special medical purposes according to claim 2, wherein the setting a size of a group and a number of iterations, determining a crossover rate and a mutation strategy, and performing optimization calculation on a digestion condition parameter combination by the digestion parameter optimization algorithm to seek for an optimal digestion condition parameter combination for each digestion stage comprise the following steps:
S131, setting a size of a group and a number of iterations, determining a crossover rate and a mutation strategy, and designing a fitness function based on a key index;
S132, converting the digestion condition parameter in a binary encoding mode, and randomly generating a parameter combination set of an initial digestion condition;
S133, calculating a fitness value of each digestion condition parameter combination, and selecting the optimal digestion condition parameter combination according to the fitness value;
S134, performing a crossover operation on the optimal digestion condition parameter combination to generate a new digestion condition parameter combination, and performing a mutation operation on the new digestion condition parameter combination; and
S135, repeatedly executing the selecting, crossover, and mutation operations, and terminating an optimization process till a preset number of iterations is reached or the fitness meets a preset condition to obtain the optimal digestion condition parameter combination for each digestion stage.

5. The combined in vitro prediction method for a glycemic index of liquid foods for special medical purposes according to claim 4, wherein the performing a crossover operation on the optimal digestion condition parameter combination to generate a new digestion condition parameter combination, and performing a mutation operation on the new digestion condition parameter combination further comprise:
selecting a certain quantity of optimal digestion condition parameter combinations from each generation of the digestion condition parameter combination set, and directly reserving the optimal digestion condition parameter combinations in a new digestion condition parameter combination set; and
dividing a total digestion condition parameter combination set into a plurality of digestion condition parameter combination subsets, setting a rule, and allowing migration of the digestion condition parameter combinations among the plurality of digestion condition parameter combination subsets.

6. The combined in vitro prediction method for a glycemic index of liquid foods for special medical purposes according to claim 1, wherein the detecting a generation quantity of glucose in the liquid food sample for special medical purposes in real time by a glucose analyzer to predict an in vitro GI value of the liquid food sample for special medical purposes comprises the following steps:
S21, placing the liquid food sample for special medical purposes in a reactor, adding a stirring rotor in the reactor, and setting corresponding stirring temperature and speed according to the parameters in the optimal digestion condition;
S22, simulating the digestion condition by using an artificial gastrointestinal simulator, wherein the artificial gastrointestinal simulator automatically adds α-amylase, pepsin, a sodium hydroxide solution, an acetic acid solution, and a trypsin mixed solution at intervals into the reactor filled with the liquid food sample for special medical purposes in sequence to react to obtain digestive juice, the trypsin mixed solution comprising trypsin and amyloglucosidase; and
S23, detecting a glucose content in the digestive juice by the glucose analyzer, and calculating the in vitro GI value of the liquid food sample for special medical purposes by built-in software of the glucose analyzer.

7. The combined in vitro prediction method for a glycemic index of liquid foods for special medical purposes according to claim 6, wherein the trypsin mixed solution comprises the trypsin and the amyloglucosidase;
a concentration of the α-amylase is 100-1000 U/ml; and/or,
a concentration of the pepsin is 1-10 mg/ml; and/or,
a concentration of the trypsin is 1-10 mg/ml; and/or,
a concentration of the amyloglucosidase is 1-10 mg/ml; and/or,
a concentration of the sodium hydroxide solution is 1-5 mol/L; and/or,
a concentration of the acetic acid solution is 1-5 mol/L;
an additive amount of the α-amylase is 1-5 mL; and/or,
an additive amount of the pepsin is 1-10 mL; and/or,
an additive amount of the trypsin mixed solution is 1-10 mL; and/or,
an additive amount of the sodium hydroxide solution is 1-10 ml; and/or,
an additive amount of the acetic acid solution is 10-30 mL.

8. The combined in vitro prediction method for a glycemic index of liquid foods for special medical purposes according to claim 1, wherein the detecting a degree of hydrolysis of starch in the sample in real time through a 3,5-dinitrosalicylic acid method to predict a theoretical GI value of the liquid food sample for special medical purposes comprises the following steps:
S31, drawing a standard curve: taking D-glucose anhydrous as a standard substance, adding water to dissolve the standard substance and dilute to a certain volume to obtain a mass concentration of the standard substance, then adding water with different volumes to dilute the standard substance to obtain solutions with different mass concentrations, taking the solutions with different mass concentrations, adding a 3,5-dinitrosalicylic acid reagent respectively, shaking the solutions well, performing color development by a water bath, cooling the solutions, adding water to dilute to a certain volume, and detecting an absorbance, taking distilled water as a blank control, to obtain the standard curve taking the mass concentration as a horizontal coordinate and the absorbance as a vertical coordinate;
S32, simulating the digestion: placing the liquid food sample for special medical purposes in a reactor, adding a stirring rotor in the reactor, setting stirring temperature and speed according to the parameters in the optimal digestion condition, adding α-amylase and oral digestive juice, performing constant temperature water bath oscillation, then adding pepsin, adjusting a pH value of the digestive juice with a hydrochloric acid solution, adding gastric digestive juice, performing constant temperature water bath oscillation, then adding trypsin, adjusting a pH value with sodium hydroxide solution, adding intestinal digestive juice, performing constant temperature water bath oscillation to obtain the digestive juice, taking the digestive juice at different time points, adding the 3,5-dinitrosalicylic acid reagent, detecting an absorbance, and obtaining a mass concentration of glucose in the digestive juice through the absorbance and the standard curve; and
S33, determining a total starch content in the sample and calculating a theoretical GI value result: placing the liquid food sample for special medical purposes in a centrifuge tube, adding the hydrochloric acid solution, performing water bath and cooling, adjusting a pH value, filtering the solution, taking a filtrate, adding the 3,5-dinitrosalicylic acid reagent, detecting an absorbance, obtaining a mass concentration of glucose in the sample after starch is hydrolyzed through the absorbance and the standard curve, and then calculating and obtaining the theoretical GI value according to a formula.

9. The combined in vitro prediction method for a glycemic index of liquid foods for special medical purposes according to claim 8, wherein the oral digestive juice comprises components with the following concentrations: 15-16 mM KCl, 3-4 mM KH₂PO₄, 13-14 mM NaHCO₃, 0.1-0.2 mM MgCl₂ (H₂O)₆, 0.01-0.10 mM (NH₄)₂CO₃, and 1-2 mM CaCl₂) (H₂O)₂; and/or,
the gastric digestive juice comprises components with the following concentrations: 5-10 mM KCl, 0.5-1.5 mM KH₂PO₄, 20-30 mM NaHCO₃, 40-50 mM NaCl, 0.1-0.3 mM MgCl₂ (H₂O)₆, 0.1-1.0 mM (NH₄)₂CO₃, and 0.1-0.2 mM CaCl₂) (H₂O)₂; and/or,
the intestinal digestive juice comprises components with the following concentrations: 5-10 mM KCl, 0.1-1 mM KH₂PO₄, 50-100 mM NaHCO₃, 30-50 mM NaCl, 0.1-1.0 mM MgCl₂ (H₂O)₆, and 0.1-1.0 mM CaCl₂) (H₂O)₂;
a concentration of the α-amylase is 50-200 U/ml; and/or,
a concentration of the pepsin is 1000-3000 U/ml; and/or,
a concentration of the trypsin is 50-200 U/ml; and/or,
a concentration of the sodium hydroxide solution is 0.1-2.0 M; and/or,
a concentration of the hydrochloric acid solution is 0.1-2.0 M;
an additive amount of the α-amylase is 1-5 mL; and/or,
an additive amount of the pepsin is 1-10 ml; and/or,
an additive amount of the trypsin is 10-20 mL.

10. The combined in vitro prediction method for a glycemic index of liquid foods for special medical purposes according to claim 8, wherein in step S33,
a calculation formula for hydrolysis rate of starch (HRS) is as follows:
$$HRS = [ ( {m}_{1} \times 0.9 ) / m ] \times 100 ,$$
where m is the total starch content, in mg; m₁ is an amount of glucose in the digestive juice, in mg; and 0.9 is a conversion coefficient for glucose and starch;
a hydrolysis curve is drawn taking time as a horizontal coordinate and the HRS as a vertical coordinate, a hydrolysis index (HI) of starch in the sample during digestion is calculated, and a calculation formula for the HI of starch in the sample during digestion is as follows:
$$HI = ( {AUC}_{sample} / {AUC}_{glucose standard substance} ) \times 100 ;$$
where AUCₛₐₘₚₗₑ is an area under a starch hydrolysis curve of the sample, and AUC_{glucose standard substance} is an area under a standard starch hydrolysis curve; and
a calculation formula for the glycemic index (GI) is as follows: GI=39.71+0.549×HI.
